(19) United States
(12) Patent Application Publication
(10) Pub. No.: US 2022/0364028 A1
(43) Pub. Date: Nov. 17, 2022

(54) SCALABLE PRODUCTION AND CULTIVATION SYSTEMS FOR PHOTO SYNTHETIC MICROORGANISMS

(71) Applicant: YEMOJA LTD., M.P. Upper Galilee (IL)

(72) Inventors: Erez ASHKENAZI, M.P. Upper Galilee (IL); Amikam BARGIL, Karmiel (IL); Moshe AVRON, Petach Tikva (IL)

(73) Assignee: YEMOJA LTD., M.P. Upper Galilee (IL)

(21) Appl. No.: 17/423,137

(22) PCT Filed: Jan. 14, 2020
(86) PCT No.: PCT/IL2020/050058

§ 371 Date: Jul. 15, 2021

Related U.S. Application Data
(60) Provisional application No. 62/792,539, filed on Jan. 15, 2019.

(51) Int. Cl. C12M 1/00; C12M 1/34; C12M 1/12; C12N 1/12

(52) U.S. Cl. CPC C12M 21/02; C12M 31/02; C12M 23/58; C12M 41/06; C12M 41/12; C12M 23/06; C12M 47/10; C12N 1/12

(57) ABSTRACT

Provided herein scalable system and processes for cultivation and production of photo synthetic microorganisms.

### TECHNICAL FIELD OF THE INVENTION

The present disclosure generally relates to the field of devices, systems and methods for cultivation and production of photosynthetic microorganisms. More particularly, the present invention relates to systems and methods for large-scale production of micro-algae.

### BACKGROUND OF THE INVENTION

Photosynthetic microorganisms, and particularly microalgae are utilized as a valuable resource for various bioactivity substances such as proteins, amino acids, carbohydrates, vitamins, antibiotics, unsaturated fatty acids, polysaccharides, and colorants. Some micro-algae species are known to produce hydrocarbon, and thus have promising application in the field of renewable energy. Today, as global food and energy crises are becoming more severe, development and utilization of micro-algal resources have exhibited a great significance and economic prospect.

A typical production process of microalgae may include cultivation of the microalgae to commercial size bulk and manipulation of the bulk under stress conditions to induce production of the desired molecule/product. Current methods for large-scale production are based on growing photosynthetic microorganisms in land-based open ponds or raceways systems that provide similar growing conditions to those found in nature. A significant drawback of this approach is inability to control the growth conditions and to ensure uniformity resulting in variable production outputs, batch contaminations and subsequent economical losses. Providing a universal, easy-to-use scalable system for large-scale production on photosynthetic organism is thus remains a long and unmet need.

### SUMMARY OF THE INVENTION

Accordingly, it is a principal object of the present invention to provide a universal, well-controlled, scalable, easy-to-use, and cost-effective systems and methods for production of photosynthetic microorganisms.

The invention provides a scalable vertical unit for cultivating a photosynthetic micro-organism comprising:
a) at least one sealable photobioreactor;
b) a column operatively engaged with the photobioreactor; and, c) at least one light source attached to the column; wherein the at least one light source and the column are aligned along the longitudinal axis of the photobioreactor; and, wherein the column is configured to control the parameters comprising temperature in the photobioreactor; the intensity of the light emitted by the light source; duration of the illumination by the light source; frequency of illumination; and, wavelength of the light emitted by the light source.

The invention further provides large-scale system for production of photosynthetic micro-organism, comprising at least two vertical cultivation units, each unit comprises a) four sealable photobioreactors; b) a column operatively engaged with the photobioreactors; c) four light sources, each operatively engaged with the column; wherein each light source and the column are aligned along the longitudinal axis of each photobioreactor and, wherein the column is configured to control the temperature in the photobioreactor, the intensity of the light emitted by the light source, frequency of illumination by the light source, duration pf the illumination, and the wavelength of the light emitted by the light source; and, wherein the first light source is aligned along the longitudinal axis of the first photobioreactor; the second light source is aligned along the longitudinal axis of the second photobioreactor; the third light source is aligned along the longitudinal axis of the third photobioreactor; the fourth light source is aligned along the longitudinal axis of the fourth photobioreactor; and, wherein the light emitted by the first light source substantially illuminates the first photobioreactor without illuminating the second, the third or the fourth photobioreactor; the light emitted by the second light source substantially illuminates the second photobioreactor without illuminating the first, the third or the fourth photobioreactor; the light emitted by the third light source substantially illuminates the third photobioreactor without illuminating the first, the second or the fourth photobioreactor; and the light emitted by the fourth light source substantially illuminates the fourth photobioreactor without illuminating the first, the second or the third photobioreactor.

The invention further provides process for large-scale production of a photosynthetic organism, comprising:
a) Providing a large-scale system for production of photosynthetic micro-organism of any one of claims comprising plurality of vertical cultivation units, each unit comprises a) four sealable photobioreactors;
b) a column operatively engaged with each of the photobioreactors; c) at least four light sources, each operatively engaged with the column; wherein each light source and the column are aligned along the longitudinal axis of each of the photobioreactors; and, wherein the column is configured to control the temperature in the photobioreactor, the intensity of the light emitted by the light source, frequency of illumination by the light source, duration of the illumination by the light source, and wavelength of the light emitted by the light source; and, wherein the light emitted by the first light source substantially illuminates the first photobioreactor without illuminating the second, the third or the fourth photobioreactor; the light emitted by the second light source substantially illuminates the second photobioreactor without illuminating the first, the third or the fourth photobioreactor; the light emitted by the third light source substantially illuminates the third photobioreactor without illuminating the first, the second or the fourth photobioreactor; and the light emitted by the fourth light source substantially illuminates the fourth photobioreactor without illuminating the first, the second or the third photobioreactor; and, d) at least one control unit in communication with each column;
b) Introducing an inoculum of the photosynthetic microorganism to the photobioreactor;
c) Adjusting parameters selected from the group consisting of temperature, light intensity; light wavelength; fluid content; nutrients; pH; gas content; and turbulence in the photobioreactor;
d) Optionally, measuring biomass in the photobioreactor; and,
e) Collecting the photosynthetic microorganism.

The invention further provides a process of obtaining at least one biomolecule produced by a photosynthetic microorganism comprising
a) Providing a large-scale system for production of the photosynthetic micro-organism of any one of claims 15 to 30;
b) Growing the photosynthetic micro-organism in the large-scale system for production of the photosynthetic micro-organism to obtain a biomass of a desired volume;
c) Optionally, inducing production of the biomolecule by the photosynthetic micro-organism to obtain biomass enriched with said at least one biologically active substance;
d) Collecting the biomass and/or growth media from the system; and,
e) Obtaining the at least one biomolecule Additional features and advantages of the invention will become apparent from the following drawings and description.

The invention further provides process of obtaining a biomass of a photosynthetic microorganism, wherein said biomass is enriched with at least one biomolecule, the process comprising:
a) providing a large-scale system for production of the photosynthetic micro-organism of any one of claims 15 to 30;
b) Growing the photosynthetic micro-organism in the large-scale system for production of the photosynthetic micro-organism of any one of claims 15 to 30 to obtain a biomass of a desired volume;
c) Optionally, inducing production of the biomolecule by the photosynthetic micro-organism to obtain biomass enriched with said at least one biomolecule; and,
d) Collecting the biomass.

Additional features and advantages of the invention will become apparent from the following drawings and description.

### BRIEF DESCRIPTION OF THE DRAWINGS

FIG. 1 (A, B) is a schematic illustration of an exemplary embodiment of a vertical scalable unit for cultivation of photosynthetic microorganisms;

FIG. 2 is a schematic illustration of an exemplary embodiment of a large-scale system for production of photosynthetic microorganisms;

FIG. 3 is a flowchart representing an exemplary embodiment of a process for large-scale production of a photosynthetic microorganism;

FIG. 4 is a flowchart representing an exemplary embodiment of a process for obtaining at least one biomolecule produced by a photosynthetic microorganism comprising; and

FIG. 5 is a flowchart representing an exemplary embodiment of a process for obtaining a biomass of a photosynthetic microorganism.

### DETAILED DESCRIPTION OF THE INVENTION

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

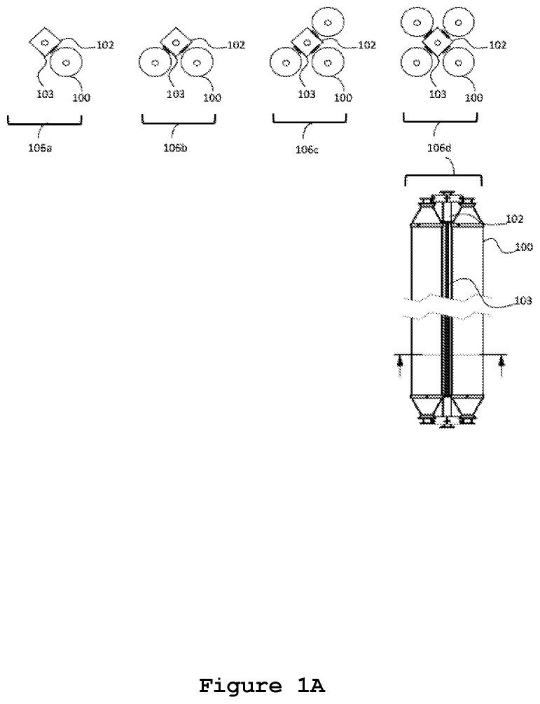

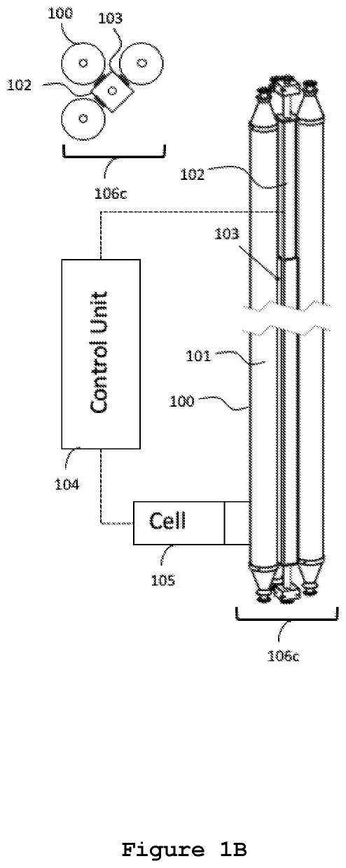

Reference is made to FIG. 1A demonstrating an exemplary embodiment of a scalable vertical unit for cultivating a photosynthetic microorganism 106a-106d. In one embodiment, the scalable vertical unit for cultivating a photosynthetic micro-organism 106a comprises a) at least one sealable photobioreactor 100; b) a column 102 operatively engaged with the at least one photobioreactor 100; c) at least one light source 103a operatively engaged with the column 102; wherein the light source 103 and the column 102 are aligned along the longitudinal axis of the photobioreactor 100. As used herein the phrase “light source operatively engaged with the column” is meant to refer, without limitation, to the light source being attached to the surface of the column, either directly or indirectly; or being embedded in the column; or being connected to a portion of the column. The contact between the light source and the column may be continuous, or alternatively, only a portion of the light source may be attached to the column. As used herein the term “photobioreactor” refers, without limitation to a bioreactor that utilizes a light source to cultivate phototrophic microorganisms that use photosynthesis to generate biomass from light and carbon dioxide. Within the artificial environment of a photobioreactor, specific conditions are carefully controlled for respective species allowing higher growth rates and purity levels than anywhere in nature or habitats similar to nature. In one embodiment, the scalable vertical unit 106b comprises two photobioreactors 100 and two light sources 103, each light source operatively engaged with the column 102; wherein the first light source is aligned along the longitudinal axis of the first photobioreactor, and the second light source is aligned along the longitudinal axis of the second photobioreactor; and wherein the light emitted by the first light source substantially illuminates the first photobioreactor without illuminating the second photobioreactor; and the light emitted by the second light source substantially illuminates the second photobioreactor without illuminating the first photobioreactor. In another embodiment, the scalable vertical unit 106c comprises three photobioreactors 100 and three light sources 103, each light source attached to the column 102; wherein the first light source is aligned along the longitudinal axis of the first photobioreactor; the second light source is aligned along the longitudinal axis of the second photobioreactor; the third light source is aligned along the longitudinal axis of the third photobioreactor; and wherein the light emitted by the first light source substantially illuminates the first photobioreactor without illuminating the second or third photobioreactor; and the light emitted by the second light source substantially illuminates the second photobioreactor without illuminating the first or the third photobioreactor; and the light emitted by the third light source substantially illuminates the third photobioreactor without illuminating the first or the second photobioreactor. According to one embodiment, the scalable vertical unit 106d comprises four photobioreactors 100 and four light sources 103, each light source attached to the column 102; wherein the first light source is aligned along the longitudinal axis of the first photobioreactor; the second light source is aligned along the longitudinal axis of the second photobioreactor; the third light source is aligned along the longitudinal axis of the third photobioreactor; the fourth light source is aligned along the longitudinal axis of the fourth photobioreactor; and wherein the light emitted by the first light source substantially illuminates the first photobioreactor without illuminating the second, the third or the fourth photobioreactor; and the light emitted by the second light source substantially illuminates the second photobioreactor without illuminating the first, the third or the fourth photobioreactor; and the light emitted by the third light source substantially illuminates the third photobioreactor without illuminating the first, the second or the fourth photobioreactor; and the light emitted by the fourth light source substantially illuminates the fourth photobioreactor without illuminating the first, the second or the third photobioreactor. According to one embodiment, the column 102 is configured to control multiple parameters in the photobioreactor 100. The non-limiting list of the parameters that may be controlled by the column includes: temperature in the photobioreactor; the intensity of the light emitted by the light source; duration of the illumination by the light source; frequency of illumination; and, wavelength of the light emitted by the light source. In one embodiment, the photobioreactor 100 comprises at least one fluid inlet; at least one fluid outlet; at least one gas inlet; at least one gas outlet; and, optionally, a cell 105 connected to the photobioreactor configured to allow collecting data related to the photobioreactor function or photobioreactor contents. Reference is now made to FIG. 1B. In one embodiment, the scalable vertical unit comprises a control unit 104 in communication with the column 102. In one embodiment the cell 105 is configured to transmit the data related to the photobioreactor function or photobioreactor contents to the control unit 104. In yet another embodiment the control unit 104 is configured to regulate the conditions inside and/or outside of the photobioreactor according to the data transmitted by the cell 105. In one embodiment, the data related to the photobioreactor function or photobioreactor contents may be, without limitation: pH; temperature; dissolved O₂ level; dissolved CO₂ level; biomass; concentration of biomolecules; concentration of nutrients; concentration of contaminants; pigment or colors. According to one embodiment, the housing 101 of the photobioreactor permits penetration of light or may otherwise incorporate a light source to provide photonic energy input for an aqueous culture of photosynthetic microorganisms. In one embodiment, the housing 101 of the photobioreactor 100 may be made, without limitation, of flexible film; a rigid thermoplastic material and/or any other material suitable for cultivating photosynthetic microorganisms. A non-limiting list of the parameters that may be regulated by the control unit include: dissolved O₂ level, dissolved CO₂ level, temperature, illumination, gas supply, mixing, pH, applied shear forces, etc.,

In one embodiment, the light source comprises a plurality of light emitting units configured to emit light of similar or different wavelengths. In another embodiment, the light emitting units of the light source are configured to emit light of 280-1000 nm. In one embodiment, the light emitting units of the light source are arranged in groups, and wherein each group of light emitting units is configured to emit light of different wavelengths. As used herein, the phrase “arranged in groups” refers, without limitation, to two or more light emitting units configured to emit light of a specific wavelength or a range of wavelengths placed in certain order within the light source. In one embodiment, at least one group of light emitting units of the light source is configured to emit photosynthetically active radiation (PAR). As used herein, the term “Photosynthetically active radiation” refers to the spectral range (wave band) of radiation from 400 to 700 nanometers that photosynthetic organisms are able to use in the process of photosynthesis. A non-limiting example of the light source of the invention is a tube or pipe containing a plurality of light emitting units In one embodiment, the light emitting unit may be, without limitation, a ballast, a fluorescent light; a light emitting diode (LED), a laser, a halogen; a neon; and an optical fiber. In another embodiment, the light source is light emitting diode (LED). Optionally, the light source includes dedicated LEDs suited for each individual type of photosynthetic organism cultivation. In a non-limiting example, each of the photobioreactor is equipped with a light source such as a LED projector line. Each of the light sources may provide the exact amount of photosynthetically active radiation (PAR), at the same angle from the same distance to keep the same lighting conditions for each photobioreactor. According to one embodiment, the non-limiting list of photosynthetic microorganisms includes marine eukaryote microalgae; marine prokaryotic microalgae; Cyanobacteria; blue/green algae; fresh-brakish water eukaryotic microalgae; halophilic eukaryotic microalgae; extremophilic eukaryotic microalgae; plants cell-lines; plants stem cells; and non-attached macroalgae (seaweeds). In one embodiment, the photosynthetic microorganism is micro-algae.

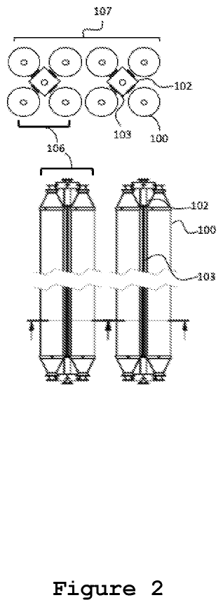

Reference is now made to FIG. 2 demonstrating an exemplary embodiment of a large-scale system for production of photosynthetic micro-organism 107. The large-scale system for production of photosynthetic micro-organism comprises at least two vertical cultivation units 106, each unit comprises a) four sealable photobioreactors 100; b) a column 102 operatively engaged with the photobioreactors 100; c) four light sources 103, each operatively engaged with the column 102; wherein each light source and the column are aligned along the longitudinal axis of each photobioreactor and, wherein the column is configured to control the temperature in the photobioreactor, the intensity of the light emitted by the light source, frequency of illumination by the light source, duration of the illumination, and the wavelength of the light emitted by the light source; and, wherein the first light source is aligned along the longitudinal axis of the first photobioreactor; the second light source is aligned along the longitudinal axis of the second photobioreactor; the third light source is aligned along the longitudinal axis of the third photobioreactor; the fourth light source is aligned along the longitudinal axis of the fourth photobioreactor; and, wherein the light emitted by the first light source substantially illuminates the first photobioreactor without illuminating the second, the third or the fourth photobioreactor; the light emitted by the second light source substantially illuminates the second photobioreactor without illuminating the first, the third or the fourth photobioreactor; the light emitted by the third light source substantially illuminates the third photobioreactor without illuminating the first, the second or the fourth photobioreactor; and the light emitted by the fourth light source substantially illuminates the fourth photobioreactor without illuminating the first, the second or the third photobioreactor. In one embodiment, each photobioreactor comprises at least one fluid inlet; at least one fluid outlet; at least one gas inlet; at least one gas outlet; and, optionally, a cell connected to the photobioreactor configured to allow collecting data related to the photobioreactor function or photobioreactor contents. In one embodiment, the production system further comprising at least one control unit in communication with each column. In another embodiment, more than one column is in communication with a single control unit. In one embodiment, the light source comprises a plurality of light emitting units configured to emit light of similar or different wavelengths. In one embodiment, the light emitting units of the light source are configured to emit light of 280-1000 nm. In another embodiment, the light emitting units of the light source are arranged in groups, and each group of light emitting units is configured to emit light of different wavelengths. According to one embodiment, at least one group of light emitting units of the light source is configured to emit photosynthetically active radiation (PAR). According to one embodiment, each of the four light sources is may be controlled independently of each other by the column and to perform differently or similarly at the same time. In one embodiment the light emitting unit is selected from the group consisting of a ballast, a fluorescent; a light emitting diode (LED), a laser, a halogen; a neon; and an optical fiber. In one embodiment, the light emitting unit is a light emitting diode (LED). According to one embodiment, the non-limiting list of photosynthetic microorganisms includes: marine eukaryote microalgae; marine prokaryotic microalgae; Cyanobacteria; blue/green algae; fresh-brakish water eukaryotic microalgae; halophilic eukaryotic microalgae; extremophilic eukaryotic microalgae; plants cell-lines; plants stem cells; and non-attached macroalgae (seaweeds). In one embodiment, the photosynthetic microorganism is micro-algae. In one embodiment, the production system comprises 10 to 10,000 vertical cultivation units. In another embodiment. In another embodiment, the production system comprises 20 to 10,000; 50 to 10,000; 100 to 10,000; 150 to 10,000; 200 to 10,000; 300 to 10,000; 400 to 10,000; 500 to 10,000; 600 to 10,000; 700 to 10,000; 800 to 10,000; 1000 to 10,000; 1,500 to 10,000; 2,000 to 10,000; and 5,000 to 10,000 vertical cultivation units. In another embodiment, the production system comprises 50 to 1000; 100 to 1,000; 150 to 1,000; 200 to 1,000; 300 to 1,000; 400 to 1,000; and 500 to 1,000 vertical cultivation units. The multiple cultivation units may be arranged for parallel/simultaneous operation. Optionally, cultivation units configured for parallel operation may be individually operated. This facilitates continuous operation of the basic unit and/or the production unit, while suspending operation of at least one of the PBRs such as for a recovery/maintenance period (e.g., clean-in-place) or due to contamination of the suspended PBR. As used herein, the term “clean-in-place,” refers to a mechanism, which can be automated, for cleaning the PBR unit without disassembly of the system/device. The term is abbreviated as “CIP”. According to one embodiment, the volume of each photobioreactor is from 5 to 100 liter. According to another embodiment, the volume of the photobioreactor is 5 to 50 liters. According to one embodiment, the volume of the photobioreactor is 15 to 35 liters. According to another embodiment, the volume of the photobioreactor is about 5; 10; 15; 20; 25; 30; 35; 40; 45; and 50 liters.

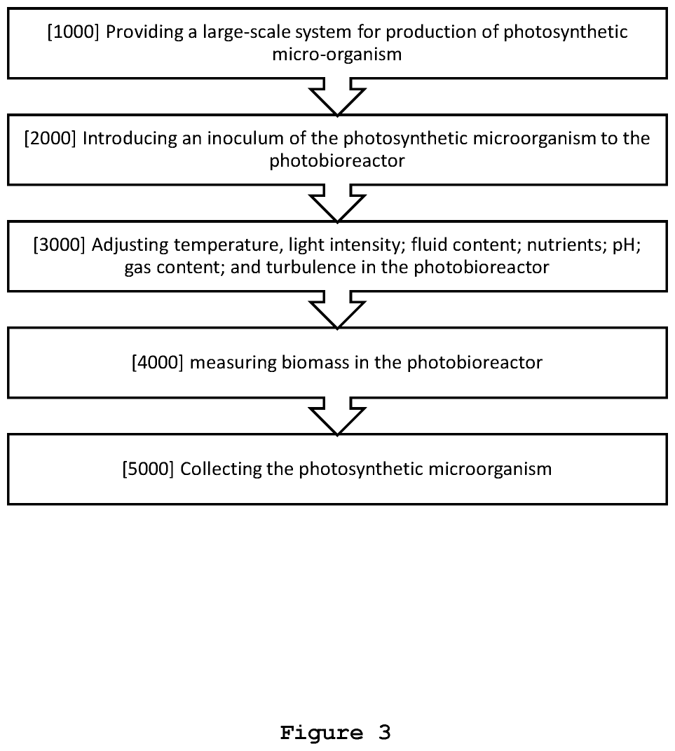

Reference is now made to FIG. 3 demonstrating an exemplary embodiment of a process for large-scale production of a photosynthetic microorganism comprising: providing a large-scale system for production of photosynthetic micro-organism [1000]; Introducing an inoculum of the photosynthetic microorganism to the photobioreactor [2000]; Adjusting parameters selected from the group consisting of temperature, light intensity; light wavelength; fluid content; nutrients; pH; gas content; and turbulence in the photobioreactor [3000]; optionally, measuring biomass in the photobioreactor [4000]; and, collecting the photosynthetic microorganism [5000]. In one embodiment, large-scale system for production of photosynthetic micro-organism comprises plurality of vertical cultivation units, each unit comprises a) four sealable photobioreactors; b) a column operatively engaged with each of the photobioreactors; c) at least four light sources, each operatively engaged with the column; wherein each light source and the column are aligned along the longitudinal axis of each of the photobioreactors; and, wherein the column is configured to control the temperature in the photobioreactor, the intensity of the light emitted by the light source, frequency of illumination by the light source, duration of the illumination episode by the light source, a number of illumination episodes, and wavelength of the light emitted by the light source; and, wherein the light emitted by the first light source substantially illuminates the first photobioreactor without illuminating the second, the third or the fourth photobioreactor; the light emitted by the second light source substantially illuminates the second photobioreactor without illuminating the first, the third or the fourth photobioreactor; the light emitted by the third light source substantially illuminates the third photobioreactor without illuminating the first, the second or the fourth photobioreactor; and the light emitted by the fourth light source substantially illuminates the fourth photobioreactor without illuminating the first, the second or the third photobioreactor; and, d) at least one control unit in communication with each column. In one embodiment, the process for large-scale production of a photosynthetic microorganism further comprises the step of collecting the growth media from the bioreactor. In one embodiment, the process for large-scale production of a photosynthetic microorganism further comprises the steps of collecting data related to photobioreactor contents or photobioreactor function; and, communicating the collected data to the control unit. As used herein, the phrase “substantially illuminates” is meant to refer to a situation when most of the light emitted by one light source in the vertical cultivation unit of the invention is directed to the corresponding PBR without illuminating the other PBRs in unit. In the context of the invention, some leakage of the light emitted by the light source toward other PBRs in the unit may occur. According to some embodiments, 1% to 50% of the light emitted by the light source toward the corresponding PBR can leak towards one or more other PBRs in the vertical cultivation unit of the invention. to thereby maintain optimal conditions for large-scale production of the photosynthetic microorganism. The cultivation/production conditions are independently controlled within each of the multiple PBR units. In one embodiment, similar conditions are independently maintained in each PBR. In another embodiment, the maintained conditions are controllably changed during the cultivation/production stages. In another embodiment, the maintained conditions are controllably adopted for cultivation/production of the desired photosynthetic microorganism species.

According to some embodiments, each light source of the vertical unit may comprise a plurality of light emitting units. The light emitting units may be arranged in groups and/or may be located separately within the light source. According to some embodiments, each group of the light emitting units comprises light emitting units configured to emit light of a specific wavelength or a range of wavelengths. The column may control each group of the light emitting units independently of one another to emit light for a desired time interval and/or intensity. According to some embodiments, the light emitting units and/or groups of light emitting units are arranged within the light source according to a desired geometry. According to some embodiments, the column controls an individual light source to generate a desired pattern of illumination by activating specific groups and/or individual light emitting units for desired time intervals and/or with desired intensity.

According to some embodiments, each light source within the vertical cultivation unit can illuminate the corresponding PBR independently of the other light sources of the unit with a desired pattern of illumination. According to some embodiments, each of the fluid inlets of the photobioreactor and the fluid outlets may be independently equipped with a valve such as a check valve and/or electronically controlled valve for introducing and releasing fluids, respectively. According to one embodiment, each of the fluid inlets and fluid outlets may be independently equipped with a pump for pumping fluids into or from the PBR, respectively. Optionally, the fluid inlet is for introducing liquids and/or gas into the photobioreactor. Optionally, the fluid outlet is for releasing liquids and/or gas from the photobioreactor. Optionally, the photobioreactor is equipped with a gas outlet and a liquid outlet. Optionally one embodiment, the turbulence element is selected from a stirrer, a mixer, a circular pumping, introduction of gas bubbles, and any combination thereof. In one embodiment, fluids removed from a PBR via fluids outlets comprises liquid and/or gas. In one embodiment, the cultivation units may be positioned in an array such as in a layer of 5-10,000 vertical cultivation units.

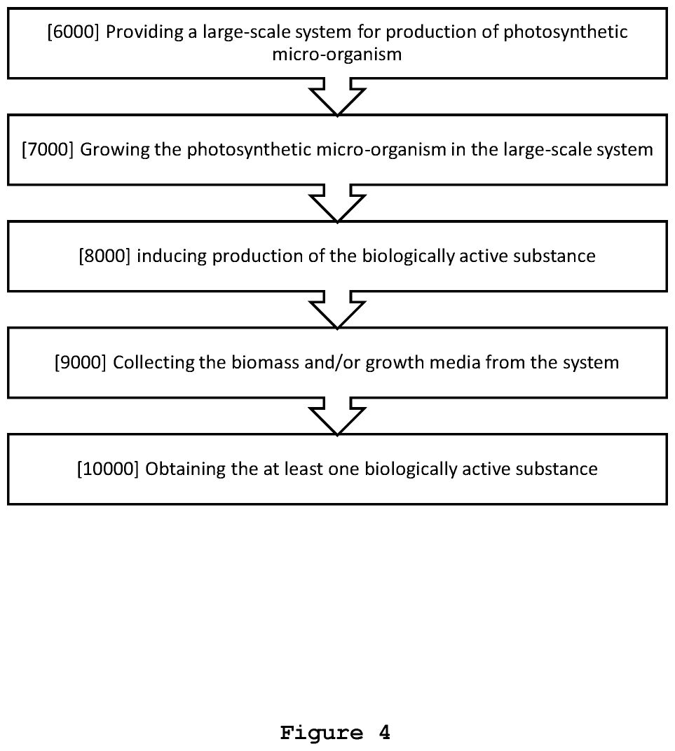

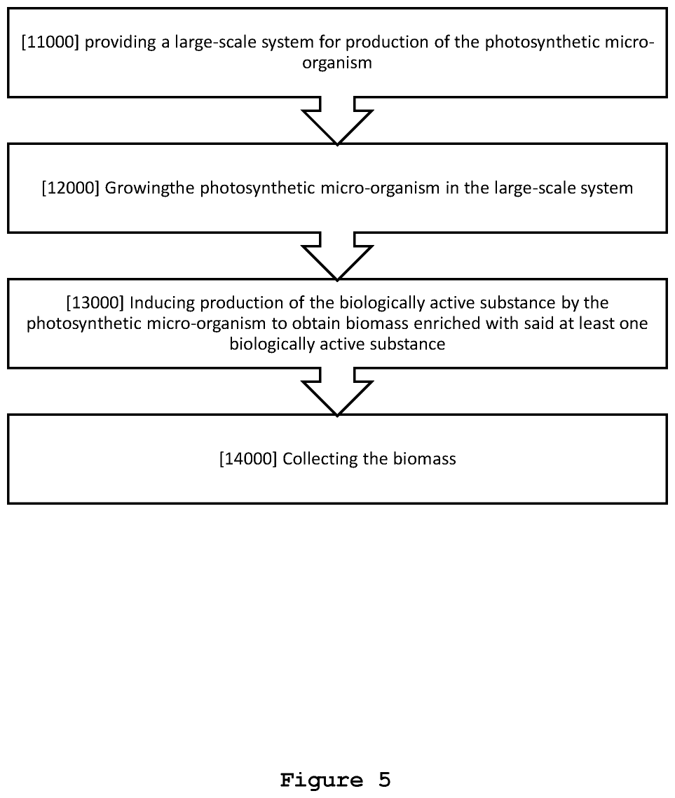

Reference is now made to FIG. 4 demonstrating an exemplary embodiment of a process of obtaining at least one biomolecule produced by a photosynthetic microorganism comprising: Providing a large-scale system for production of the photosynthetic micro-organism of the invention [6000]; Growing the photosynthetic micro-organism in the large-scale system for production of the photosynthetic micro-organism to obtain a biomass of a desired volume [7000]; optionally, inducing production of the biomolecule by the photosynthetic micro-organism to obtain biomass enriched with the at least one biomolecule [8000]; collecting the biomass and/or growth media from the system [9000]; and, obtaining the at least one biomolecule [10000]. In one embodiment, the biomolecule is secreted by the photosynthetic micro-organism into the growth media. In another embodiment, the biomolecule is obtained from the biomass. In one embodiment, the biomolecule can be obtained from the biomass by the means of, without limitation, extraction, separation or any other techniques known in the art for such purposes. In one embodiment, a mixture of biomolecules is obtained by the process. According to some embodiments, the non-limiting list of biomolecules includes: alkaloids, flavonoids, carotenoids, glycosides, terpenoids, phenazines, proteins, peptides, polypeptides, vitamins, carbohydrates, lipids, polysaccharides, polyols, phycobiliproteins, cellulose, hemicellulose, pectin, lipopolysaccharides, chlorophyll, fatty acids, lipids, oils, saccharides, glycerides, poly-glycerides, quinones, lignans, polyions, pigments and chelators. According to some embodiments, the biomolecules can have biological effect. According to some embodiments, the biomolecules may act as antioxidant; bio-stimulants; crop protection agents; anti-aging agents; anti-inflammatory agents; anti-viral agents; and, antibiotics. According to some embodiments, the biomolecules produced by the photosynthetic microorganisms of the invention can be used, without limitation as pharmaceuticals, nutraceuticals, cosmeceuticals, food supplements, agrochemicals, perfumes, in a textile industry and as plant growth regulators. Reference is now made to FIG. 5, demonstrating an exemplary embodiment of process of obtaining a biomass of a photosynthetic microorganism enriched with at least one biomolecule comprising: [11000]; growing the photosynthetic micro-organism in the large-scale system for production of the photosynthetic micro-organism to obtain a biomass of a desired volume [12000]; optionally, inducing production of the biomolecule by the photosynthetic micro-organism to obtain biomass enriched with said at least one biomolecule [13000]; and collecting the biomass [14000]. As used herein the phrase “inducing production of biomolecule” refers, without limitation, to applying conditions that facilitate production and/or secretion of the biomolecule and/or activating biological pathway leading to de-novo synthesis of the biomolecule by the photosynthetic micro-organism. According to some embodiment, conditions that induce production of biomolecule include, without limitation, temperature, illumination, and nutrient supply. According to some embodiments, stress conditions such as non-optimal temperature, irradiation by UV, or any other stress conditions known in the art that may lead to the induction of production of biomolecules.

According to some embodiments, following propagation of the photosynthetic microorganism, a purification step may be carried out to separate the biomass, which can be used for extracting additional products or sold as high value feed. Optionally, the purified product (e.g., biomass and/or extracts thereof), may be further subjected to pasteurization/sterilization.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that when an element is referred to as being “on,” “attached” to, “operatively coupled” to, “operatively linked” to, “operatively engaged” with, “connected” to, “coupled” with, “contacting,” etc., another element, it can be directly on, attached to, connected to, operatively coupled to, operatively engaged with, coupled with and/or contacting the other element or intervening elements can also be present. In contrast, when an element is referred to as being “directly contacting” another element, there are no intervening elements present.

Whenever the term “about” is used, it is meant to refer to a measurable value such as an amount, a temporal duration, and the like, and is meant to encompass variations of ±20%, +10%, +5%, +1%, or ±0.1% from the specified value, as such variations are appropriate to perform the disclosed methods.

It will be understood that, terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Whenever terms “plurality” and “a plurality” are used it is meant to include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

All publications, patent applications, patents, and other references mentioned. The disclosures of these publications in their entireties are hereby incorporated by reference into this application in order to more fully describe the state of the art to which this invention pertains. In case of conflict, the patent specification, including definitions, will prevail. In addition, the materials, methods, and examples are illustrative only and not intended to be limiting. Throughout this application various publications, published patent applications and published patents are referenced.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

1. A scalable vertical unit for cultivating a photosynthetic micro-organism comprising:
a) at least one sealable photobioreactor;
b) a column operatively engaged with the photobioreactor; and,
c) at least one light source operatively engaged with the column; wherein the at least one light source and the column are aligned along the longitudinal axis of the photobioreactor; and, wherein the column is configured to control the parameters comprising temperature in the photobioreactor; the intensity of the light emitted by the light source; duration of the illumination by the light source; frequency of illumination; and, wavelength of the light emitted by the light source.

2. The scalable vertical unit of claim 1, wherein the photobioreactor comprises at least one fluid inlet; at least one fluid outlet; at least one gas inlet; at least one gas outlet; and, optionally, a cell connected to the photobioreactor configured to allow collecting data related to the photobioreactor function or photobioreactor contents; and, wherein the scalable vertical unit optionally comprises a control unit in communication with the column.

3. (canceled)

4. The scalable vertical unit of claim 1, comprising two, three or four photobioreactors and two, three or four light sources, each light source operatively engaged with the column; wherein the first light source is aligned along the longitudinal axis of the first photobioreactor, the second light source is aligned along the longitudinal axis of the second photobioreactor; the third light source is aligned along the longitudinal axis of the third photobioreactor; the fourth light source is aligned along the longitudinal axis of the fourth photobioreactor; and wherein the light emitted by the first light source substantially illuminates the first photobioreactor without illuminating the second, the third or the fourth photobioreactor; the light emitted by the second light source substantially illuminates the second photobioreactor without illuminating the first the third or the fourth photobioreactor; the light emitted by the third light source substantially illuminates the third photobioreactor without illuminating the first, the second or the fourth photobioreactor; and the light emitted by the fourth light source substantially illuminates the fourth photobioreactor without illuminating the first, the second or the third photobioreactor.

5. (canceled)

6. (canceled)

7. The scalable vertical unit of claim 1, wherein the light source comprises a plurality of light emitting units configured to emit light of similar or different wavelengths; and wherein the light emitting units of the light source are optionally arranged in groups, wherein each group of the light emitting units is configured to emit light of different wavelength.

8. The scalable vertical unit of claim 7, wherein the light emitting units of the light source are configured to emit light of 280-1000 nm; and, optionally, wherein at least one light emitting unit of the light source is configured to emit photosynthetically active radiation (PAR).

9. (canceled)

10. (canceled)

11. The scalable vertical unit of claim 7, wherein the light emitting unit is selected from the group consisting of a ballast, a fluorescent, a light emitting diode (LED), a laser, a halogen, a neon, and an optical fiber.

12. (canceled)

13. The scalable vertical unit of claim 1, wherein the photosynthetic organism is selected form the group consisting of marine eukaryote microalgae; marine prokaryotic microalgae; Cyanobacteria; blue/green algae; fresh-brakish water eukaryotic microalgae; halophilic eukaryotic microalgae; extremophilic eukaryotic microalgae; plants cell-lines; plants stem cells; and non-attached macroalgae (seaweeds).

14. (canceled)

15. A large-scale system for production of photosynthetic micro-organism, comprising at least two vertical cultivation units, each unit comprises a) four sealable photobioreactors; b) a column operatively engaged with the photobioreactors; c) four light sources, each operatively engaged with the column; wherein each light source and the column are aligned along the longitudinal axis of each photobioreactor and, wherein the column is configured to control the temperature in the photobioreactor, the intensity of the light emitted by the light source, frequency of illumination by the light source, duration pf the illumination, and the wavelength of the light emitted by the light source; and, wherein the first light source is aligned along the longitudinal axis of the first photobioreactor; the second light source is aligned along the longitudinal axis of the second photobioreactor; the third light source is aligned along the longitudinal axis of the third photobioreactor; the fourth light source is aligned along the longitudinal axis of the fourth photobioreactor; and, wherein the light emitted by the first light source substantially illuminates the first photobioreactor without illuminating the second, the third or the fourth photobioreactor; the light emitted by the second light source substantially illuminates the second photobioreactor without illuminating the first, the third or the fourth photobioreactor; the light emitted by the third light source substantially illuminates the third photobioreactor without illuminating the first, the second or the fourth photobioreactor; and the light emitted by the fourth light source substantially illuminates the fourth photobioreactor without illuminating the first, the second or the third photobioreactor.

16. The production system of claim 15, wherein each photobioreactor comprises at least one fluid inlet; at least one fluid outlet; at least one gas inlet; at least one gas outlet; and, optionally, a cell connected to the photobioreactor configured to allow collecting data related to the photobioreactor function or photobioreactor contents; and, wherein at least one of the at least two vertical cultivation units optionally comprises at least one control unit in communication with the column.

17. (canceled)

18. The production system of claim 15, wherein the light source comprises a plurality of light emitting units configured to emit light of similar or different wavelengths; and wherein the light emitting units of the light source are optionally arranged in groups, and wherein each group of light emitting units is configured to emit light of different wavelengths.

19. The production system of claim 18, wherein the light emitting units of the light source are configured to emit light of 280-1000 nm; and, optionally, wherein at least one light emitting unit of the light source is configured to emit photosynthetically active radiation (PAR).

20. (canceled)

21. (canceled)

22. The production system of claim 18, wherein the light emitting unit is selected from the group consisting of a ballast, a fluorescent; a light emitting diode (LED), a laser, a halogen; a neon; and an optical fiber.

23. (canceled)

24. The production system of claim 15, wherein the photosynthetic organism is selected from the group consisting of marine eukaryote microalgae; marine prokaryotic microalgae; Cyanobacteria; blue/green algae; fresh-brakish water eukaryotic microalgae; halophilic eukaryotic microalgae; extremophilic eukaryotic microalgae; plants cell-lines; plants stem cells; and non-attached macroalgae (seaweeds).

25. (canceled)

26. The production system of claim 15, comprising 10 to 10,000 vertical cultivation units.

27. (canceled)

28. (canceled)

29. The production system of claim 15, wherein the volume of each photobioreactor is 5 to 50 liters.

30. (canceled)

31. A process for large-scale production of a photosynthetic microorganism, comprising:
a) Providing a large-scale system for production of photosynthetic micro-organism of claim 15;
b) Introducing an inoculum of the photosynthetic microorganism to the photobioreactor;
c) Adjusting parameters selected from the group consisting of temperature, light intensity; light wavelength; fluid content; nutrients; pH; gas content; and turbulence in the photobioreactor;
d) Optionally, measuring biomass in the photobioreactor;
e) Collecting the photosynthetic microorganism; and, optionally,
f) collecting the growth media from the bioreactor.

32. (canceled)

33. (canceled)

34. (canceled)

35. (canceled)

36. A process of obtaining at least one biomolecule produced by a photosynthetic microorganism comprising
a) Providing a large-scale system for production of the photosynthetic micro-organism of claim 15;
b) Growing the photosynthetic micro-organism in the large-scale system for production of the photosynthetic micro-organism to obtain a biomass of a desired volume;
c) Optionally, inducing production of the biomolecule by the photosynthetic micro-organism to obtain biomass enriched with said at least one biomolecule;
d) Collecting the biomass and/or growth media from the system; and,
e) Obtaining the at least one biomolecule.

37. The process of claim 36, wherein the biomolecule is obtained from the growth media or from the biomass; and wherein the biomolecule is selected from the group consisting of alkaloids, flavonoids, carotenoids, glycosides, terpenoids, phenazines, proteins, peptides, polypeptides, vitamins, carbohydrates, lipids, polysaccharides, polyols, phycobiliproteins, cellulose, hemicellulose, pectin, lipopolysaccharides, chlorophyll, fatty acids, lipids, oils, saccharides, glycerides, poly-glycerides, quinones, lignans, polyions, and chelators.

38. (canceled)

39. (canceled)

40. (canceled)

41. (canceled)

42. A process of obtaining a biomass of a photosynthetic microorganism,
wherein said biomass is enriched with at least one biomolecule, the process comprising:
a) providing a large-scale system for production of the photosynthetic micro-organism of claim 15;
b) Growing the photosynthetic micro-organism in the large-scale system for production of the photosynthetic micro-organism to obtain a biomass of a desired volume;
c) Optionally, inducing production of the biomolecule by the photosynthetic micro-organism to obtain biomass enriched with said at least one biomolecule; and
d) Collecting the biomass.
